(19) United States
(12) Patent Application Publication
(10) Pub. No.: US 2005/0255085 A1
(43) Pub. Date: Nov. 17, 2005

(54) Prevention of recurrence and metastasis of cancer

(76) Inventors: Yuman Fong, New York, NY (US); Richard Wong, New York, NY (US)

Correspondence Address: CLARK & ELBING LLP, 101 FEDERAL STREET, BOSTON, MA, 02110, US

(21) Appl. No.: 10/505,375

(22) PCT Filed: Mar. 3, 2003
(86) PCT No.: PCT/US03/06519

§ 371 Date: May 10, 2005

Related U.S. Application Data
(60) Provisional application No. 60/361,132, filed on Mar. 1, 2002.

(51) Int. Cl. A61K 48/00; C12N 15/86

(52) U.S. Cl. 424/93.2; 435/456; 424/93.6

(57) ABSTRACT

The invention provides methods of preventing and treating cancer, involving the use of attenuated, replication-com-petent, oncolytic herpes viruses.

### FIELD OF THE INVENTION

This invention relates to methods of preventing and treating cancer.

### BACKGROUND OF THE INVENTION

The impact of cancer on our society cannot be overstated. Cancer is the second leading cause of death in the United States, being surpassed only by heart disease. Indeed, 1 in 4 deaths in the United States is caused by cancer (American Cancer Society, Cancer Facts and Figures 2001, New York 2001, ACS, Inc.).

A cell becomes cancerous when its normal growth control mechanisms become impaired. At first, the uncontrolled growth of cancerous cells is confined to the tissue in which the cells originated but, over time, the cells can spread, or metastasize, from their site of origin to another area of the body. For example, cancer cells may infiltrate the walls of blood or lymph vessels, thus entering the circulatory or lymphatic systems, from which they may lodge in another tissue and seed the growth of secondary, metastatic tumors. It is thought that fewer than 1 in 10,000 cells that are shed from a primary tumor actually survive, but this small portion of surviving cells is sufficient to seed secondary tumors elsewhere in the body.

About 35% of patients that are newly diagnosed with cancer lack metastases, and these patients can be cured by localized treatment of their tumors by, e.g., surgery or radiation. The remaining patients either already have detectable metastases (about 30%) or have undetectable metastases that will eventually develop into tumors (about 35%). Treatment of these patients often involves a systemic approach such as, for example, the administration of chemotherapeutic drugs that interfere with the growth of rapidly dividing cells, such as cancer cells. The overall five-year relative survival rate of all cancers is only 60%, which underscores the importance of early detection, enabling tumor treatment (e.g., removal) before metastasis occurs, as well as the development of therapeutic approaches to treating or, preferably, preventing cancer metastasis.

### SUMMARY OF THE INVENTION

The invention provides methods of preventing or treating cancer in a subject, e.g., a human subject. The methods involve surgical resection of a tumor from the subject, followed by administration of an attenuated, replication-competent, oncolytic herpes virus by, for example, injection into the site of surgical resection. Alternatively, the virus can be injected into the tumor directly, which may then, optionally, be resected. The invention also includes the use of an attenuated, replication-competent, oncolytic herpes virus (e.g., HSV-1) in the preparation of medicaments for carrying out these methods. The administered herpes virus prevents or treats the recurrence of any cancer that may remain at the site of resection, as well as prevents or treats any cancer that may have metastasized from the site of surgical resection. The metastasized cancer may be found in the lymphatic system, for example, in a lymph node.

Herpes viruses that can be used in the methods of the invention include herpes simplex virus-1 (HSV-1)-derived viruses, e.g., NV1023. Optionally, the herpes virus administered according to the methods of the invention includes a heterologous nucleic acid molecule encoding a therapeutic product, which can be, for example, a cytotoxin, an immunomodulatory protein, a tumor antigen, an antisense nucleic acid molecule, or a ribozyme. The methods of the invention can also include the use of a second (or more) anticancer treatment. For example, the methods can be carried out in conjunction with chemotherapy, biological therapy, radiation therapy, or gene therapy.

The invention provides several advantages. For example, when the virus is administered after surgical removal of gross disease, it has as its target only microscopic residual tumor, rather than a large tumor volume, enabling more concentrated, efficient delivery. Also, as is shown in the experiments described below, the virus has oncolytic activity when injected directly into tumors. Thus, the methods of the invention can be used to treat primary tumors, as well as to prevent lymphatic metastases. The herpes viruses administered according to the methods of the invention follow the same pathways as metastasizing tumor cells, thus enhancing the likelihood of their reaching those areas within the lymphatic system, e.g., lymph nodes, that are at greatest risk for harboring metastatic disease. An additional advantage of the methods of the invention is that they employ mutant herpes viruses that replicate in, and thus destroy, dividing cells, such as; cancer cells, while not affecting other, quiescent cells in the body. The herpes viruses can also be multiply mutated, thus eliminating the possibility of reversion to wild type. Moreover, if necessary, the replication of the herpes viruses can be controlled through the action of antiviral drugs, such as acyclovir, which block viral replication, thus providing another important safeguard. An additional advantage of using replication-competent viruses is that only a fraction of tumor cells need to be infected initially, before the viruses propagate in permissive cancerous tissue. The invention thus provides targeted, safe, and effective methods for preventing and treating primary site cancer recurrences, as well as regional lymphatic metastases.

Other features and advantages of the invention will be apparent from the following detailed description, the drawings, and the claims.

### BRIEF DESCRIPTION OF THE DRAWINGS

FIGS. 1A, 1B, 1C, and 1D: FIG. 1A is a photograph showing a mouse that has been injected with blue dye at the base of the posterior auricle. FIG. 1B is a photograph showing that injection of the blue dye results in rapid blue color detection in an ipsilateral cervical lymph node. The normal lymphatic drainage pattern of the murine auricle leads to the ipsilateral cervical lymph nodes. FIGS. 1C and 1D are photographs showing the development of metastatic disease within these same cervical lymph nodes 2 weeks (1C) and 4 weeks (1D) after implantation of squamous cell carcinoma (SCC) VII tumors into the auricle. Approximately 20% of mice implanted with SCC VII tumors will demonstrate cervical metastases upon neck exploration.

FIGS. 2A and 2B are photographs showing that the implantation and growth of auricular SCC VII tumors results in histological evidence of metastases to the draining cervical lymph nodes. FIG. 2A is an H&E stain of a cervical lymph node showing SCC VII cells first infiltrating the subcapsular sinus (100×). FIG. 2B is a higher power view of another H&E stained lymph node section showing metastatic SCC VII cells adjacent to normal lymphocytes (800×).

FIGS. 3A, 3B, and 3C are photographs showing that virally infected cells may be detected histologically in the draining cervical lymph nodes following auricular injections of oncolytic herpes virus. FIG. 3A shows that NV1023 (2×10⁷ pfu) injected into the left auricle results in scattered lacZ-expressing blue cells detected at 24 hours in the ipsilateral cervical lymph nodes. FIG. 3B shows a DAPI-stained nodal section in which NV1066 (2×10⁷ pfu) injected into the left auricle can be observed under fluorescence microscopy in the ipsilateral cervical lymph nodes by examination at 24 hours. The DAPI stain is used to visualize all nuclei. FIG. 3C shows cells from an adjacent cervical lymph node section that have been infected with NV1066, which promotes expression of the green fluorescent protein.

FIG. 4 is a graph demonstrating the reduction of average auricular tumor volumes due to intratumoral injections of NV1023. Established auricular tumors 6-8 mm in dimension were treated with three serial intratumoral injections (days 0, 2, and 4) of NV1023 (2×10⁷ pfu). Average auricular tumor volumes were significantly reduced for the virally treated group at day 7 compared to the PBS treated group (p<0.0001, t-test).

FIGS. 5A and 5B are photographs showing that metastatic deposits of SCC VII within the cervical lymph nodes are successfully infected by NV1023 delivered to the surgical beds of excised auricular tumors. FIG. 5A is a photograph showing an H&E stained section from excised cervical nodes that demonstrates complete replacement with metastatic SCC VII cells (400×). FIG. 5B is a photograph showing an adjacent nodal section stained with X-gal that demonstrates scattered blue-staining metastatic SCC VII cells, reflecting infection by NV1023 (400×).

FIG. 6 is a graph showing that metastatic tumor volume in the cervical lymph nodes is reduced with NV1023 treatment at the primary site. Auricular tumors were excised and the surgical beds treated with 5×10⁷ pfu of NV1023. Average cervical nodal volumes were lower for the virally treated group compared to the PBS treated group (days 6-15).

FIG. 7 is a graph showing that disease free survival is significantly improved with NV1023 treatment (5×10⁷ pfu) of the surgical bed following resection of auricular SCC VII tumors (p<0.05, log rank test).

### DETAILED DESCRIPTION

The invention provides methods of preventing and treating cancer. In the methods of the invention, a tumor is surgically removed from a subject and the site of the resection is treated with an attenuated, replication competent, oncolytic herpes virus. Alternatively, the virus can be injected directly into a tumor, which may then, optionally, be resected. As is noted above, such viruses selectively replicate in, and thus destroy, cancer cells, while leaving non-cancerous cells unharmed. The administered herpes virus thus eliminates any microscopic disease remaining at the site of resection, thereby preventing recurrence at that site. The administered herpes virus also enters the lymphatic system from the site of the primary tumor in the same manner as any potentially metastasizing tumor cells, thus enabling the treatment and prevention of metastasis from the primary tumor site. Use of these viruses in the methods of the invention, as well as experimental results showing the efficacy of these methods, are described further below.

Cancers

Examples of cancers that can be prevented or treated using the methods of the invention include skin (e.g., squamous cell carcinoma, basal cell carcinoma, or melanoma), breast, colorectal, prostate, brain and nervous system, head and neck, testicular, ovarian, pancreatic, lung, liver (e.g., hepatoma), kidney, bladder, gastrointestinal, bone, endocrine system (e.g., thyroid and pituitary tumors), and lymphatic system (e.g., Hodgkin's and non-Hodgkin's lymphomas) cancers. Cancers of the nervous-system include, for example, astrocytoma, oligodendroglioma, meningioma, neurofibroma, glioblastoma, ependymoma, Schwannoma, neurofibrosarcoma, neuroblastoma, and medulloblastoma. Other types of cancers that can be treated using the methods of the invention include fibrosarcoma, neuroectodermal tumor, mesothelioma, epidermoid carcinoma, as well as any other cancers that form solid tumors.

Viruses

Viruses that can be used in the methods of the invention can be derived from any of the members of the family Herpesviridae. For example, herpes simplex virus-1 (HSV-1)-derived viruses can be used. Additional examples of herpes family viruses from which viruses that are used in the invention can be derived are herpes simplex virus-2 (HSV-2), vesicular stomatitis virus (VSV), cytomegalovirus (CMV), Epstein-Barr virus (EBV), human herpes virus-6 (HHV-6), human herpes virus-7 (HHV-7), and human herpes virus-8 (HHV-8). A central feature of the viruses that can be used in the methods of the invention is that they are replication-competent, and thus are able to infect, replicate in, and lyse malignant cells, while at the same time they are sufficiently attenuated to not adversely affect normal cells.

Two specific examples of HSV-1-derived viruses that can be used in the methods of the invention are NV1023 (Wong et al., Hum. Gene Ther. 12:253-265, 2001) and NV1020, which are described in further detail below. An additional specific example of an HSV-1-derived virus that can be used in the invention is G207 (Yazaki et al., Cancer Res. 55(21):4752-4756, 1995). This virus has deletions in both copies of the γ34.5 gene, as well as an inactivating insertion in UL39, which is the gene that encodes infected-cell protein 6 (ICP6), the large subunit of HSV ribonucleotide reductase.

Still a further specific example of a herpes virus that can be used in the invention is G47Δ(Todo et al., Proc. Natl. Acad. Sci. U.S.A. 98(11):6396-6401, 2001), which is a multimutated, replication-competent HSV-1 vector that was derived from G207 by a 312 basepair deletion within the non-essential α47 gene (Mavromara-Nazos et al., J. Virol. 60:807-812, 1986). Because of the overlapping transcripts encoding ICP47 and US11 in HSV, the deletion in α47 places the late US11 gene under control of the immediate-early α47 promoter, which enhances the growth properties of γ34.5⁻ mutants.

Additional examples of attenuated HSV viruses that can be used in the methods of the invention include hrR3, which is ribonucleotide reductase-defective (Spear et al., Cancer Gene Ther. 7(7):1051-1059, 2000), HF (ATCC VR-260), Maclntyre (ATCC VR-539), MP (ATCC VR-735), HSV-2 strains G (ATCC VR-724) and MS (ATCC VR-540), as well as any viruses having mutations (e.g., inactivating mutations, deletions, or insertions) in any one or more of the following genes: the immediate early genes ICP0, ICP22, and ICP47 (U.S. Pat. No. 5,658,724); the Δ34.5 gene; the ribonucleotide reductase gene; and the VP16 gene (i.e., Vmw65, WO 91/02788, WO 96/04395, and WO 96/04394). The vectors described in U.S. Pat. Nos. 6,106,826 and 6,139,834, as well as other replication-competent, attenuated herpes viruses, can also be used in the methods of the invention.

The effects of the viruses used in the methods of the invention can be augmented, if desired, by including heterologous nucleic acid sequences encoding one or more therapeutic products in the viruses. For example, nucleic acid sequences encoding cytotoxins, immunomodulatory proteins (i.e., proteins that enhance or suppress patient immune responses to antigens), tumor antigens, antisense RNA molecules, or ribozymes can be included in the viruses. Examples of immunomodulatory proteins that can be encoded by the heterologous nucleic acid sequences include, e.g., cytokines (e.g., interleukins, for example, any of interleukins 1-15, α, β, or γ-interferons, tumor necrosis factor (TNF), granulocyte macrophage colony stimulating factor (GM-CSF), macrophage colony stimulating factor (M-CSF), and granulocyte colony stimulating factor (G-CSF)), chemokines (e.g., neutrophil activating protein (NAP), macrophage chemoattractant and activating factor (MCAF), RANTES, and macrophage inflammatory peptides MIP-1a and MIP-1b), complement components and their receptors, immune system accessory molecules (e.g., B7.1 and B7.2), adhesion molecules (e.g., ICAM-1, 2, and 3), and adhesion receptor molecules. Appropriate heterologous nucleic acid sequences for use in the methods of the invention can be readily selected by those of skill in this art.

The heterologous nucleic acid sequences can be inserted into the viruses for use in the methods of the invention in a location that renders them under the control of regulatory sequences of the viruses. Alternatively, the heterologous nucleic acid sequences can be inserted as part of an expression cassette that includes regulatory elements, such as promoters or enhancers. Appropriate regulatory elements can be selected by those of skill in the art based on, for example, the desired tissue-specificity and level of expression. For example, a cell-type specific or tumor-specific promoter can be used to limit expression of a gene product to a specific cell type. This is particularly useful, for example, when a cytotoxic, immunomodulatory, or tumor antigenic gene product is being produced in a tumor cell in order to facilitate its destruction, and provides a further safeguard of specificity. In addition to using tissue-specific promoters, local (i.e., intra-resection site) administration of the viruses of the invention can result in localized expression and effect.

Tumor specific promoters can also be selected for use in the invention, based on the etiology of the cancer. Examples of promoters that function specifically in tumor cells include the stromelysin 3 promoter, which is specific for breast cancer cells (Basset et al., Nature 348:699, 1990); the surfactant protein A promoter, which is specific for non-small cell lung cancer cells (Smith et al., Hum. Gene Ther. 5:29-35, 1994); the secretory leukoprotease inhibitor (SLPI) promoter, which is specific for SLPI-expressing carcinomas (Garver et al., Gene Ther. 1:46-50, 1994); the tyrosinase promoter, which is specific for melanoma cells (Vile et al., Gene Therapy 1:307, 1994; WO 94/16557; WO 93/GB1730); the epidermal growth factor receptor promoter, which is specific for squamous cell carcinoma, glioma, and breast tumor cells (Ishii et al., Proc. Natl. Acad. Sci. U.S.A. 90:282, 1993); the mucin-like glycoprotein (DF3, MUC1) promoter, which is specific for breast carcinoma cells (Abe et al., Proc. Natl. Acad. Sci. U.S.A. 90:282, 1993); the mts1 promoter, which is specific for metastatic tumors (Tulchinsky et al., Proc. Natl. Acad. Sci. U.S.A. 89:9146, 1992); the NSE and somatostatin receptor promoters, which are specific for small-cell lung cancer cells (Forss-Petter et al., Neuron 5:187, 1990; Bombardieri et al., Eur. J. Cancer 31A:184, 1995; Koh et al., Int. J. Cancer 60:843, 1995); the c-erbB-2 promoter, which is specific for pancreatic, breast, gastric, ovarian, and non-small cell lung cells (Harris et al., Gene Ther. 1:170, 1994); the c-erbB-3 promoter, which is specific for breast cancer cells (Quin et al., Histopathology 25:247, 1994); and the c-erbB4 promoter, which is specific for breast and gastric cancer cells (Rajkumar et al., Breast Cancer Res. Trends 29:3, 1994). Examples of non-tissue specific promoters that can be used in the invention include the early Cytomegalovirus (CMV) promoter (U.S. Pat. No. 4,168,062) and the Rous Sarcoma Virus promoter (Norton et al., Mol. Cell Biol. 5:281, 1985). Also, HSV promoters, such as HSV-1 IE and IE 4/5 promoters, can be used.

Any of a number of well-known formulations for introducing viruses into cells in patients can be used in the invention. (See, e.g., Remington's Pharmaceutical Sciences (18^{th} edition), ed. A. Gennaro, 1990, Mack Publishing Co., Easton, Pa.) However, the viruses can be simply diluted in a physiologically acceptable solution, such as sterile saline or sterile buffered saline, with or without an adjuvant or carrier. The amount of virus to be administered can readily be determined by those of skill in this art, and depends on factors such as, for example, the condition of the patient intended for administration (e.g., the weight, age, and general health of the patient), the mode of administration, and the type of formulation. In general, an effective dose of, e.g., from about 10¹ to 10¹⁰ plaque forming units (pfu), for example, from about 5×10⁴ to 1×10⁶ pfu, e.g., from about 1×10⁵ to about 4×10⁵ pfu, is administered, although the most effective ranges may vary from patient to patient, as can readily be detennined by those of skill in this art.

The viruses are administered to sites of surgical resection in patients by, for example, injection directly into the surgical bed after resection of a primary tumor, either before or after closing of the surgical site. Alternatively, as is discussed above, the viruses can be injected directly into tumors.

The methods of the invention can employ replication competent, attenuated herpes viruses as sole therapeutic agents or, alternatively, these agents can be used in combination with other anticancer treatments. Examples of additional therapies that can be used include chemotherapy, biological therapy, gene therapy, radiation therapy, antisense therapy, and therapy involving the use of angiogenesis inhibitors (e.g., angiostatin, endostatin, and icon). Selection of any of these types of therapies for use with replication-competent, attenuated herpes in the methods of the invention can readily be carried out by those of skill in the art.

Specific examples of chemotherapeutic agents that can be used in the methods of the invention are provided as follows. These compounds fall into several different categories, including, for example, alkylating agents, antineoplastic antibiotics, antimetabolites, and natural source derivatives. Examples of alkylating agents that can be used in the methods of the invention include busulfan, carboplatin, carnustine, chlorambucil, cisplatin, cyclophosphamide (i.e., cytoxan), dacarbazine, ifosfamide, lomustine, mecholarethamine, melphalan, procarbazine, streptozocin, and thiotepa; examples of antineoplastic antibiotics include bleomycin, dactinomycin, daunorubicin, doxorubicin, idarubicin, mitomycin (e.g., mitomycin C), mitoxantrone, pentostatin, and plicamycin; examples of antimetabolites include fluorodeoxyuridine, cladribine, cytarabine, floxuridine, fludarabine, flurouracil (e.g., 5-fluorouracil (5FU)), gemcitabine, hydroxyurea, mercaptopurine, methotrexate, and thioguanine; and examples of natural source derivatives include docetaxel, etoposide, irinotecan, paclitaxel, teniposide, topotecan, vinblastine, vincristine, vinorelbine, taxol, prednisone, tamoxifen, asparaginase, and mitotane.

The biological therapy that can be used in the methods of the invention can involve administration of an immunomodulatory molecule, such as a molecule selected from the group consisting of tumor antigens, antibodies, cytokines (e.g., interleukins, interferons, tumor necrosis factor (TNF), granulocyte macrophage colony stimulating factor (GM-CSF), macrophage colony stimulating factor (M-CSF), and granulocyte colony stimulating factor (G-CSF)), chemokines, complement components, complement component receptors, immune system accessory molecules, adhesion molecules, and adhesion molecule receptors.

The methods of the invention, as described herein, are based, in part, on the experimental results that are described as follows.

### EXPERIMENTAL RESULTS

Summary

Oncolytic herpes viruses have significant antitumor effects in animal models when delivered directly to established tumors. Lymphatic metastases are a common occurrence for many tumor types. This study investigates the use of an attenuated, replication-competent, oncolytic herpes simplex virus (NV1023), both to treat a primary tumor by direct injection, and to travel through the lymphatic system to treat metastatic tumors within the lymph nodes draining lymph from the site of primary cancer. Isosulfan blue dye was injected into murine auricles to determine normal lymphatic drainage patterns, and demonstrated consistent blue staining of a group of ipsilateral cervical lymph nodes. Auricular injections of NV1023 resulted in viral transit to these lymph nodes, as measured by X-gal histochemistry and viral plaque assay. Using the SCC VII cell line, a novel murine model of auricular squamous cell carcinoma was developed with an approximately 20% incidence of cervical lymph node metastases. Delivery of NV1023 to surgical beds following excision of auricular SCC VII tumors resulted in successful viral infection of metastatic SCC VII cells within the cervical lymph nodes. After a 7 week follow-up, significantly enhanced locoregional control (p<0.05, Fischer's exact test) and disease free survival (p<0.05, Log rank test) were evident with NV1023 treatment. This study demonstrates that the delivery of NV1023 to a primary tumor site following surgical excision reduces both primary site recurrence and regional nodal metastases.

Lymph Node Drainage Patterns

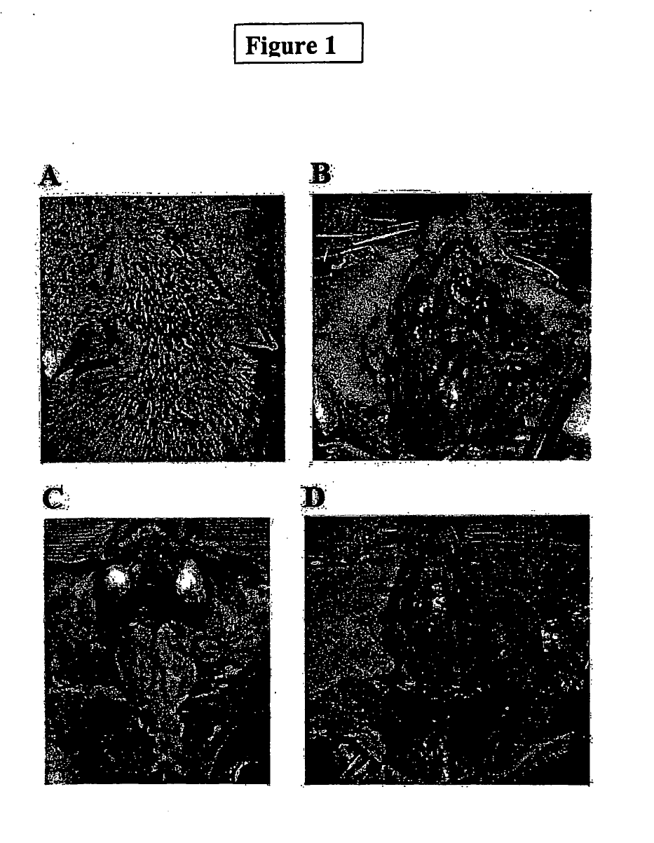

Isosulfan blue dye (100 μl) was injected into the base of the left posterior auricle to identify the normal draining lymph nodes for this anatomic site. In all cases (n=5), intense blue dye was visible within a group of 1-3 ipsilateral cervical lymph nodes adjacent to the external jugular vein and salivary gland tissue. These cervical lymph nodes were consistently identified as the primary draining nodes to the auricular region (FIGS. 1A and 1B). Contralateral cervical lymph nodes and ipsilateral nodes deep to the sternocleidomastoid muscle did not stain blue in any animal.

SCC VII Auricular-Cervical Metastatic Model

To develop a model of cervical lymphatic metastases, SCC VII tumors were implanted in the left auricles of mice and grown to 13-18 mm in largest dimension to allow for the microscopic seeding of cervical lymph nodes. The growth of these auricular tumors did not cause significant morbidity, and did not impair either feeding or respiration. The auricular tumors were then excised to control primary site morbidity, to prolong survival, and to permit the subsequent development of palpable cervical node metastases.

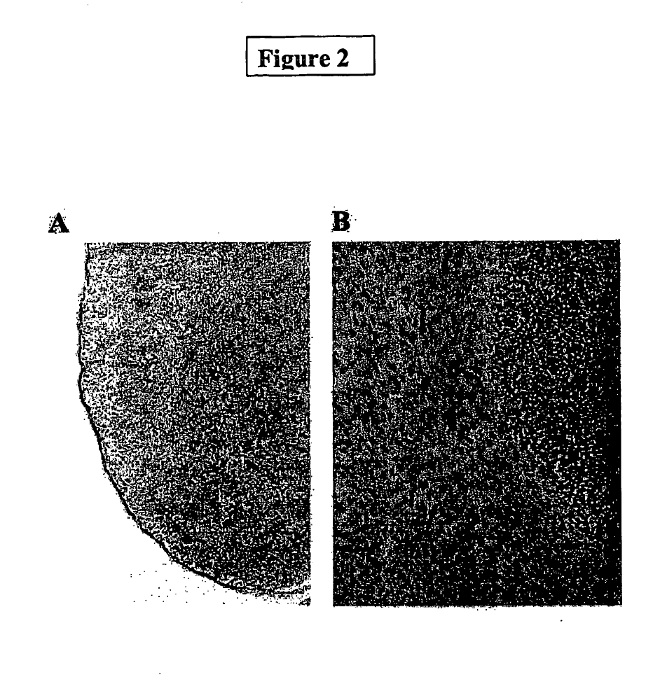

The implantation and excision of auricular SCC VII tumors in C3H/HeJ mice led to approximately 20% of these animals developing palpable adenopathy in the ipsilateral neck within the following two weeks (FIGS. 1C and 1D). Histologic examination confirmed the presence of metastatic squamous cell carcinoma in cases of palpable nodes, which were generally >8 mm in dimension. Histologic examination of excised lymph nodes demonstrated that metastatic SCC VII cells are deposited in the subcapsular sinus of the lymph node before progressively infiltrating the nodal parenchyma and replacing the entire nodal architecture (FIGS. 2A and 2B). Primary site recurrence at the sites of primary auricular tumor resection was noted in approximately 10% of cases.

Viral Transit From Auricle to Cervical Lymph Nodes

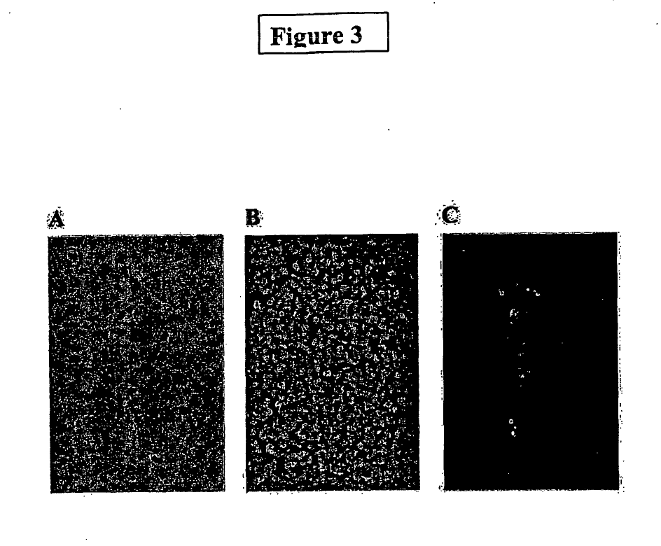

The ability of an oncolytic virus to travel from the auricle to the draining cervical lymph nodes was demonstrated by injecting NV1023 into the left auricle of non-tumor bearing animals and histologically examining the ipsilateral draining lymph nodes at 24 and 48 hours for X-gal staining cells. At 24 hours, there was positive X-gal staining within the ipsilateral cervical lymph nodes (FIG. 3A). Blue stained cells tended to be sparse and scattered. At 48 hours, most ipsilateral draining nodes were negative for blue cells. Contralateral lymph nodes at both 24 and 48 hours were negative for X-gal staining cells.

Successful viral transit from auricle to the cervical lymph nodes was further confirmed by using the GFP-expressing NV1066 virus. NV1066 was injected into the left posterior auricle and the cervical lymph nodes were harvested 24 hours later. Fluorescent microscopy of ipsilateral cervical lymph nodes demonstrated the presence of sparse, scattered green fluorescence, reflecting the presence of NV1066-infected cells (FIGS. 3B and 3C).

The number of recoverable viral plaque forming units (pfu) from the draining lymph nodes was also determined by viral plaque assay. Draining lymph nodes excised 10 minutes after auricular viral injections of NV1023 yielded approximately 5000 viral pfu/gm of nodal tissue. No live virus was recovered from any ipsilateral nodes excised 24 hours after auricular viral injection, or from any contralateral lymph nodes excised at either 10 minutes or 24 hours. This transient and sparse appearance of virus within the lymphatics of animals not bearing cancer is as would be expected from viruses designed to have limited infectivity for non-cancerous tissues.

Viral Therapy of SCC VII Auricular Tumors

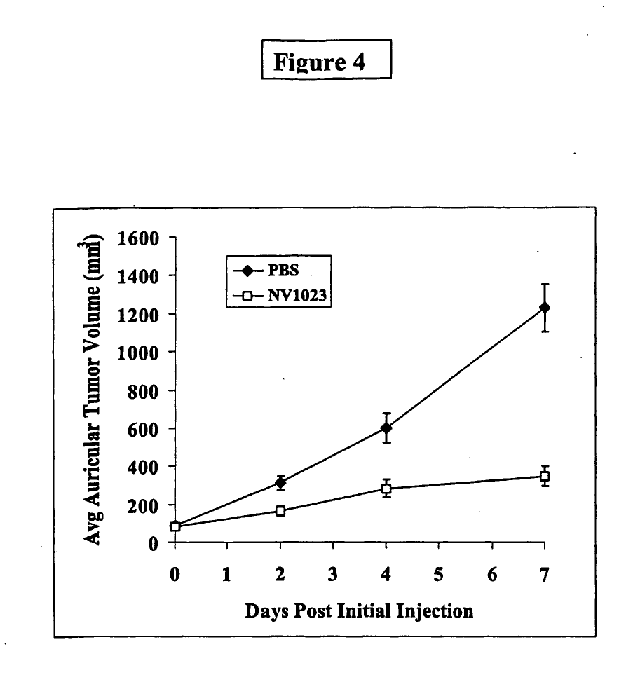

To determine the in vivo efficacy of NV1023 against established SCC VII tumors, NV1023 was injected as three serial doses into established auricular tumors and subsequent tumor dimensions recorded. Average tumor volumes for the NV1023 treated animals were significantly decreased as compared to controls (p<0.0001 at day 7, t-test, FIG. 4).

Viral Therapy of SCC VII Cervical Metastases

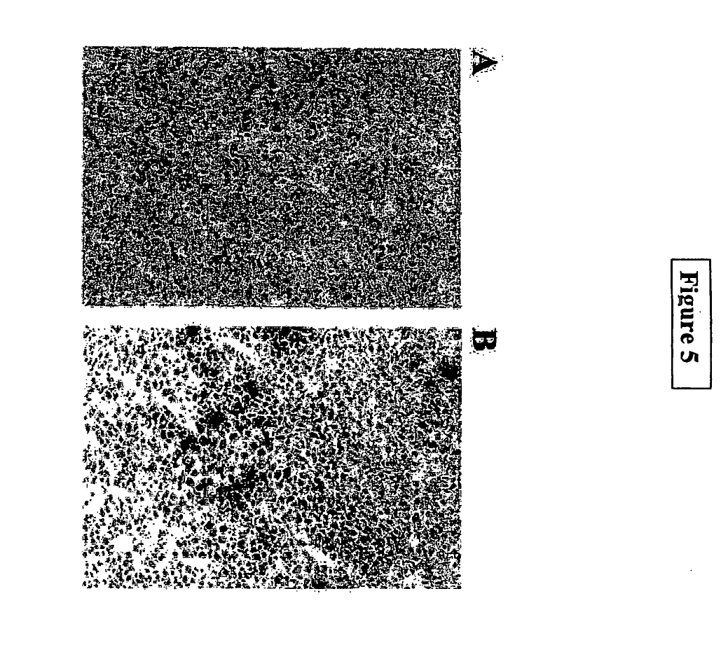

NV1023 was delivered to the surgical bed after excision of established auricular SCC VII tumors. At 24 hours after viral delivery, animals underwent neck exploration, cervical node excision, and histologic examination of bilateral nodal groups. X-gal staining revealed the presence of blue-staining metastatic SCC VII deposits within the lymph nodes (FIGS. 5A and 5B). X-gal staining was minimal in adjacent normal lymphocytes and in lymph nodes without metastatic SCC VII cells.

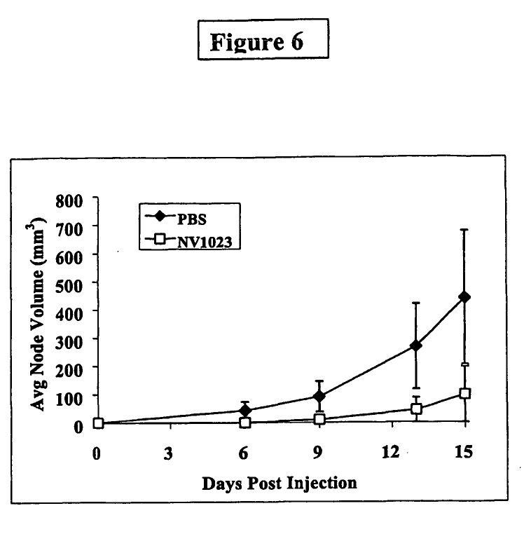

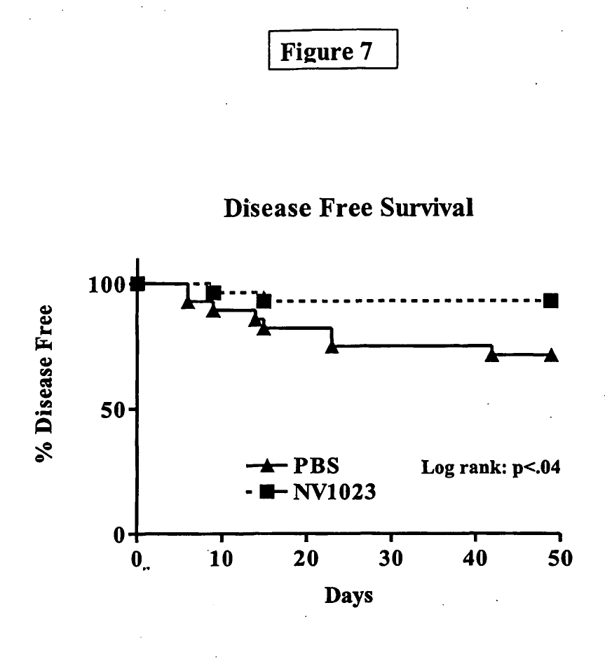

A survival experiment was performed by comparing surgical bed treatment with either PBS (n=28) or NV1023 (n=28) following the excision of auricular tumors. Animals were subsequently monitored for either primary (auricular) recurrence or the development of regional (cervical) metastases. The average cervical nodal volume of the PBS treated group (440 mm³) was greater than that of the NV1023 treated group (98 mm³) at day 15 (FIG. 6). Of the 28 animals receiving PBS, 3 (10.7%) developed primary site recurrences at the auricular excision site, and 5 (17.9%) developed palpable nodal metastases in the ipsilateral neck, for a total of 8 (28.6%) locoregional failures. Of the 28 animals receiving NV1023, 1 (3.6%) developed a primary site recurrence and 1 (3.6%) developed a palpable nodal metastasis, for a total of 2 (7.1%) locoregional failures. There were no cases of both primary site recurrence and nodal metastasis occurring within the same animal. There was also no evidence of distant metastases in either group. The NV1023-treated group showed a significantly enhanced locoregional control rate (p<0.05, Fischer's exact test) as compared to the PBS-treated control group. With a follow-up period of 7 weeks, disease free survival was significantly enhanced (p<0.05, Log rank test) for the NV1023-treated group (FIG. 7).

There was also no evidence of any morbidity resulting from NV1023 administration. There was no significant weight loss, mucosal or cutaneous ulcerations, neurotoxicity, or other toxicities detected in any of the virally treated animals. All auricular incision sites demonstrated rapid and complete wound healing following NV1023 administration to the surgical bed.

### MATERIALS AND METHODS

Cell Lines

The murine SCC VII cell line is a cutaneous squamous cell carcinoma that spontaneously arose from the C3H/HeJ mouse. SCC VII (H. Suit, Harvard University) is a rapidly dividing cell line with an estimated doubling time of 18 hours (Fu et al., Int. J. Radiat. Oncol. Biol. Phys. 10:1473-1478, 1984; O'Malley et al., Arch. Otolaryngol. Head Neck Surg. 123:20-24, 1997). SCC VII cells were grown in vitro in MEM containing 10% FCS at standard cell culture conditions. African green monkey kidney (Vero) cells for viral plaque assays were also grown in MEM containing 10% FCS at standard cell culture conditions (American Type Culture Collection, Manassas, Va.).

Viruses

NV1023 is an attenuated, replication-competent, oncolytic herpes virus whose construction has been previously described in detail (Wong et al., Hum. Gene Ther. 12:253-265, 2001). NV1023 carries a non-functional, 5.2 kb fragment of HSV-2 DNA in the U_{L/S} junction. This HSV-2 fragment was originally inserted into the NV1020 (R7020) virus, from which NV1023 was derived, to broaden its potential application as a herpes vaccine (Meignier et al., J. Infect. Dis. 158:602-614, 1988). NV1023 is attenuated by a 15 kilobase deletion in the inverted repeat U_{L/S} junction that deletes one copy of the γ₁134.5 neurovirulence gene and the UL56 gene. NV1023 also contains the E. coliβ-galactosidase (lacZ) gene inserted at the US10-12 locus to serve as a marker of infection.

NV1020 (Medigene Inc., San Diego, Calif.) is an attenuated, replication-competent derivative of herpes simplex virus type-1 (HSV-1) (Delman et al., Hum. Gene Ther. 11:2465-2472, 2000). NV1020 is a non-selected clonal derivative from R7020, a candidate HSV-1/2 vaccine strain that was obtained from Dr. B. Roizman (Meigner et al., J. Infect. Dis. 158:602-614, 1998). The structure of NV1020 is characterized by a 15 kilobase deletion encompassing the internal repeat region, leaving only one copy of the following genes, which are normally diploid in the HSV-1 genome: ICP0, ICP4, the latency associated transcripts (LATs), and the neurovirulence gene γ₁34.5. A fragment of HSV-2 DNA encoding several glycoprotein genes was inserted into this deleted region. In addition, a 700 basepair deletion encompasses the endogenous thymidine kinase (TK) locus, which also prevents the expression of the overlapping transcripts of the U_{L}24 gene. An exogenous copy of the HSV-1 TK gene was inserted under control of the α4 promotor. Virus was propagated in Vero cells and harvested by freeze thaw lysis to release virus from the cell fraction. Cell lysates were clarified by centrifugation, and viral titers were determined on Vero cells by plaque assay. All virus preparations were formulated in D-PBS-10% glycerin and stored at −80° C.

Animals

All animal procedures were approved by the Memorial Sloan-Kettering Institutional Animal Care and Use Committee. Six-week old male C3H/HeJ mice (Jackson Laboratory, Bar Harbor, Me.) were anesthetized with inhalational methoxyflurane for injections of isosulfan blue dye, SCC VII tumor cells, and NV1023 or NV1066 virus. Each animal received an intraperitoneal injection of ketamine (70 μg) and xylazine (20 μg) in 100 μl of sterile water prior to the surgical excision of auricular tumors. Animals were sacrificed by CO₂ inhalation.

Lymph Node Drainage Patterns

The normal lymphatic drainage pattern of the auricular region was determined by injecting 1% isosulfan blue dye (100 μl) into the base of the posterior left auricle of C3H/HeJ mice (n=5). At two minutes following injection, mice were sacrificed, their necks surgically explored, and the draining cervical nodes visually identified by the presence of blue dye.

Development of SCC VII Auricular-Cervical Metastatic Model

A novel head and neck metastatic model of murine squamous cell carcinoma was developed. Auricular tumors were established by the injection of 1×10⁶ SCC VII cells in 50 μl PBS into the base of the left posterior auricle of each mouse. By day 13, the auricular tumors ranged from 13-18 mm in greatest dimension. All tumors were then completely surgically excised with the left auricle, and the incision was closed with a running 4-0 nylon suture.

Over a two to three week postoperative period, animals were monitored for the subsequent development of palpable adenopathy in the ipsilateral neck. At varying time points following tumor excision, animals were sacrificed and their necks surgically explored. Enlarged cervical nodes were excised, immediately frozen in imbedding media (Tissue Tek, Sagura Inc., Torrance, Calif.), cut into 6 μm thick sections, stained with hematoxylin and eosin, and examined histologically to identify the presence of metastatic squamous cell carcinoma.

Viral Transit From Auricle to Cervical Lymph Nodes

To document the ability of virus to travel from the auricle to the cervical lymph nodes, NV1066 or NV1023 was injected at a dose of 2×10⁷ pfU/100 μl of phosphate buffered saline (PBS) into the base of the left posterior auricle in non-cancer-bearing C3H/HeJ mice. After 24 or 48 hours, mice were sacrificed and their necks surgically explored. Ipsilateral and contralateral cervical lymph nodes were excised, frozen in Tissue Tek, cut into 6 μm thick sections, mounted on glass slides, washed in PBS, and examined.

Nodes from animals injected with NV1066 and from control animals were examined under fluorescence microscopy at wavelengths from 515-585 nm, and GFP expression identified by the presence of fluorescent green color. Sections were also stained with 20 μl of 4,6-diamino-2 phenylindole (DAPI, 0.1 μg/ml) in mounting media (1 mg p-phenylenedamine/1 cc of 80% glycerol in PBS) to identify cellular nuclei by blue fluorescence.

Nodes from animals injected with NV1023 and from controls were stained with 5-bromo-4-chloro-3-indol-β-D-galactopyranoside (X-gal) at 37° C. for 2 hours, as previously described (Geller et al., Science 241:1667-1669, 1988) for assessment of β-gal expression. Counterstaining of background cell nuclei with nuclear fast red was performed. Virally infected cells expressing β-Sgalactosidase were identified histologically as blue-staining cells.

To measure viral recovery from the cervical lymph nodes, NV1023 was again injected at a dose of 2×10⁷ pfU in 100 μl of PBS into the left auricles of mice. At 10 minutes (n=3) and 24 hours (n=3) following viral injection, animals were sacrificed and the bilateral cervical lymph nodes were surgically excised, weighed, homogenized in 250 μl of PBS, mixed, and subjected to three freeze-thaw cycles to lyse cells. After a second centrifugation (30 seconds, 10,000 rpm), supernatants were collected and titered on confluent Vero cells, as previously described, to determine the quantity of viral plaque forming units recovered (Wong et al., Hum. Gene Ther. 12:253-265, 2001).

Viral Therapy of SCC VII Auricular Tumors

Auricular tumors were established by the injection of 5×10⁵ SCC VII cells in 50 μl PBS into the base of the left posterior auricle in C3H/HeJ mice. Visible tumors developed in all animals within 34 days. By day 6, tumors were approximately 5-6 mm in greatest dimension, and animals were distributed into two groups of equitable tumor volumes. One group (n=8) was treated with three serial intratumoral injections of NV1023 at 2×10⁷ pfu in 100 μl PBS (delivered every other day). The other group (n=8) received an identical regimen of PBS injections as a control. Subsequent tumor dimensions were recorded and volumes calculated by the formula for the volume of an ellipsoid: volume=(4/3)*π*(length/2)*(width/2)².

Viral Therapy of SCC VII Cervical Metastases by Injection of Primary Tumor Sites

Auricular SCC VII tumors were established as described above. On day 13 after tumor cell injection, tumor volumes were measured and animals were divided into two groups with equitable tumor volumes. Auricular tumors were completely excised in all mice. Immediately after tumor excision and wound closure with 4-0 nylon suture, one group of animals (n=28) was treated with NV1023 and the other group (n=28) with PBS. NV1023 at a dose of 5×10⁷ pfu in 100 μl PBS was injected through the closed incision line and into the potential space between the skin and the surgical bed. The control group of animals underwent identical injections of 100 μl PBS. A separate group of animals (n=10) was treated identically with NV1023, and cervical lymph nodes were subsequently excised 24 and 48 hours later and examined by histochemical staining for β-galactosidase expression.

Animals were routinely weighed and monitored postoperatively for the development of palpable cervical metastatic disease, primary site (auricular) recurrence, or any toxicity related to tumor growth or virus administration. The dimensions of any palpable cervical adenopathy that subsequently developed were measured with calipers, and nodal volumes calculated. Animals were sacrificed if the greatest nodal dimension or primary site recurrence exceeded 18 mm, if there was evidence of skin ulceration, or if there was any other morbidity evident.

All publications and patent applications mentioned in this specification are herein incorporated by reference to the same extent as if each independent publication or patent application was specifically and individually indicated to be incorporated by reference.

1. A method of preventing or treating cancer in a subject, said method comprising the steps of:
surgically resecting a tumor from the subject; and
administering an attenuated, replication-competent, oncolytic herpes virus to the site of surgical resection.

2. The method of claim 1, wherein said cancer is present at the site of surgical resection.

3. The method of claim 1, wherein said cancer has metastasized from the site of surgical resection.

4. The method of claim 3, wherein said cancer is present in the lymphatic system of said subject.

5. The method of claim 4, wherein said cancer is present in a lymph node of said subject.

6. The method of claim 1, wherein said herpes virus is a herpes simplex-1-derived virus.

7. The method of claim 6, wherein said herpes virus is NV1023.

8. The method of claim 1, wherein said subject is a human.

9. The method of claim 1, wherein said herpes virus is administered to said subject by injection.

10. The method of claim 1, wherein said herpes virus comprises a heterologous nucleic acid molecule encoding a therapeutic product.

11. The method of 10, wherein said therapeutic product is selected from the group consisting of cytotoxins, immunomodulatory proteins, tumor antigens, antisense nucleic acid molecules, and ribozymes.

12. The method of claim 1, further comprising administering a second anticancer treatment to said subject.

13. The method of claim 12, wherein said second anticancer treatment is selected from the group consisting of chemotherapy, biological therapy, radiation therapy, and gene therapy.

14. The method of claim 1, further comprising injecting an attenuated, replication-competent, oncolytic herpes virus into said tumor of said subject prior to exision.

15-21. (canceled)

22. The method of claim 14, further comprising administering a second anticancer treatment to said subject.

23. The method of claim 22, wherein said second anticancer treatment is selected from the group consisting of chemotherapy, biological therapy, radiation therapy, and gene therapy.

24-27. (canceled)
